(19) United States
(12) Patent Application Publication
(10) Pub. No.: US 2026/0363566 A1
(43) Pub. Date: Nov. 19, 2026

(54) WEARABLE DEVICE FOR PPG AND CONTACTLESS PAYMENTS

(71) Applicant: ELAD MEIR, RISHON LEZION (IL)

(72) Inventor: ELAD MEIR, RISHON LEZION (IL)

(21) Appl. No.: 19/211,332

(22) Filed: May 19, 2025

(51) Int. Cl. A61B 5/024; A61B 5/00; A61B 5/11; A61B 5/1455; A61B 5/16; G06Q 20/32

(52) U.S. Cl. CPC A61B 5/02438; A61B 5/002; A61B 5/02427; A61B 5/1118; A61B 5/14552; A61B 5/165; A61B 5/681; A61B 5/742; G06Q 20/3278; A61B 2560/0219; A61B 2562/0219

(57) ABSTRACT

A PPG device that includes at least one PPG sensor (14) designed to measure health-related characteristics of its user, a charging device to supply power to the PPG device, an output interface for transmitting sensor readings to an external device thereto, where the connectivity to external devices thereto can be carried out by NFC, where the PPG device is annular in shape and appropriately sized to wrap around the strap (16) of a wristwatch (12), ensuring direct contact with the user's body for accurate measurements.

### TECHNICAL FIELD

The invention relates to the field of measuring wearable physiological properties and for carrying out payments.

### BACKGROUND

A smartwatch is a sophisticated wearable device that blends the classic wristwatch design with advanced smart functionalities. Seamlessly connecting to your smartphone, it enables instant access to notifications, messages, and incoming calls. Beyond communication, smartwatches serve as personal wellness companions, tracking physical activity, monitoring health metrics like heart rate and sleep patterns, and even facilitating contactless payments. With a diverse range of apps, they enhance productivity and simplify daily tasks.

Unlike smartphones, smartwatches incorporate an array of sensors designed to measure and analyze physical activity and health data. Some of the most commonly used sensors include:

Advanced smartwatch models feature additional sensors, such as those for measuring blood oxygen levels (SpO2), sleep patterns, temperature fluctuations, and even PPG sensors for more precise health monitoring.

One challenge of smartwatches is their dependency on continuous wear. While they offer valuable features, many users enjoy switching up their watches for fashion and personal expression. Balancing functionality with style remains an ongoing consideration in smartwatch design, with solutions emerging to make them more adaptable and seamlessly integrated into everyday wardrobes.

All the methods described above have not yet provided satisfactory solutions to the problem of combining functionality of a wristwatch with changing fashion requirements.

### SUMMARY OF THE INVENTION

In one aspect, the invention pertains to a PPG device that includes at least one PPG sensor (14) designed to measure health-related characteristics of its user, a charging mechanism to supply power to the PPG device, an output interface for transmitting sensor readings to an external device to the PPG device, where the PPG device is annular in shape and appropriately sized to wrap around the strap (16) of a wristwatch (12), ensuring direct contact with the user's body for accurate measurements.

Additionally, the PPG device may feature a closure (15) to secure its annular form.

According to various embodiments of the invention:
The PPG sensor monitors heart pulses;
The PPG sensor assesses stress levels of the user;
The PPG sensor measures blood oxygen levels.

For data transmission, the output mechanism may utilize:
NFC communication, providing near-field connectivity;
Bluetooth communication, enabling wireless data exchange.

The PPG device can use the NFC communication for transferring PPG data out of said PPG device to an external device thereto.

The PPG device can use the NFC communication also for carrying out contactless payments to an external device thereto.

Furthermore, the PPG device may incorporate an IMU sensor, enhancing its ability to track the user's physical activity and heart health parameters.

Regarding external connectivity:
The PPG device can communicate with the user's smartphone;
Data from the PPG device can be relayed to a medical institution via the smartphone.

For power supply:
The PPG device supports wireless charging, such as according to the Qi standard;
The charging mechanism may be according to the Qi standard.

The reference numbers have been used to point out elements in the embodiments described and illustrated herein, in order to facilitate the understanding of the invention. They are meant to be merely illustrative, and not limiting. Also, the foregoing embodiments of the invention have been described and illustrated in conjunction with systems and methods thereof, which are meant to be merely illustrative, and not limiting.

### BRIEF DESCRIPTION OF THE DRAWINGS

Embodiments, features, and aspects of the invention are described herein in conjunction with the following drawings, in which:

FIG. 1 pictorially illustrates a wrist of a user wearing a wristwatch to which a PPG device is attached, according to an embodiment of the invention;

FIG. 2 schematically illustrates a PPG device, according to an embodiment of the invention;

FIG. 3 pictorially illustrates a PPG device, according an embodiment of the invention;

FIG. 4 pictorially illustrates a PPG device, according an embodiment of the invention; and

FIG. 5 schematically illustrates a PPG device 100 communication with a smartphone 20, according an embodiment of the invention.

It should be understood that the drawings are not necessarily drawn to scale.

### DETAILED DESCRIPTION

The invention will be understood from the following detailed description of embodiments which are meant to be descriptive and not limiting. For the sake of brevity, some well-known features, methods, systems, procedures, components, circuits, and so on, are not described in detail.

The followings are terms that will be used in the description:

The term sensor refers herein to a device that detects and responds to physical, chemical, or biological stimuli from its environment. It converts the detected information (i.e., the sensor reading) into an electrical signal or another form of data that can be interpreted and processed.

Sensors can measure features such as temperature, pressure, light, motion, humidity, sound, and even biometric data.

A sensor typically consists of three parts:
(a) The sensing element, which interacts with the stimulus and produces a measurable change;
(b) The transducer, which converts the measured change into an electrical signal;
(c) The signal processing unit, which refines and transmits the output for further use.
For example, a temperature sensor detects heat variations and converts them into an electrical signal, which can then be displayed on a screen or trigger an automated response in a climate control system.

The term PPG sensor (Photoplethysmography sensor) refers herein to a technology used in wearable devices such as smartwatches to measure heart rate and other health related data. These sensors may use light to measure changes in blood volume over time, through the skin. When a wearable device is on the wrist, it sends light to the skin and measures the reflection of the light, thus calculating the heart rate.

PPG sensors provide to smartwatches ability to track physical activity, report stress levels, and sometimes also measure blood oxygen levels of a user. They are an important part of the set of tools that may help users maintain fitness and health.

The term IMU (Inertial Measurement Unit) refers herein to a sensor that measures and reports an object's specific force, angular rate, and sometimes magnetic field. IMUs are used in navigation, robotics, aerospace, and various motion-tracking applications to determine position, orientation, and movement.

A smart ring is a wearable technological device in the form of a ring, which contains various sensors and electronic modules, like fitness bands and smartwatches. Smart rings are also used for health and communication purposes. Here are some of the common features of smart rings:

Fitness and health tracking: Heart rate monitoring, sleep monitoring, exercise tracking, and blood oxygen level (SpO2) measurement. Smart notifications and alerts: Receive notifications of incoming calls, text messages, and other important information via vibration or indicator lights. Security and identity verification: Use the ring as an authentication factor for devices, lock and unlock smart doors, and make secure payments. Smart device control: Control smart home devices, such as lighting, TVs, and other smart systems.

Smart rings are designed to provide technological solutions in a small and comfortable to wear form factor. They are especially suitable for those who prefer not to wear a bracelet or smartwatch, but still want to enjoy the benefits of wearable devices in context with health subject matters.

The term NFC (Near Field Communication) refers herein to a technology for wireless communication over a “very short” range, usually up to a few centimeters. The technology allows devices to communicate and exchange information with each other when they are physically close.

In everyday use, NFC is widely used in contactless payments (such as Apple Pay and Google Wallet), public transportation (smart travel cards), and data sharing between devices (such as photos and information between smartphones). The device sends low-frequency radio signals to transfer information securely and simply, so it is quick and convenient to use.

In the context of PPG (Photoplethysmography) technology, LEDs are used to measure heart rate and blood flow by shining light through the skin and analyzing the light reflected from the blood:
Green LED: Most commonly used in PPG, as hemoglobin in the blood absorbs green light optimally, allowing sensors to measure changes in blood flow with high accuracy.
Infrared LED: Used for deeper measurements, such as blood oxygen (SpO2) sensors and heart rate measurement in low light conditions or on darker skin, where light reflectance is different.
Blue LED: Less commonly used in PPG, but has some uses in medical research and specific measurements of skin properties.

The green LED is the main one for heart rate measurement, the infrared is used for depth measurement, and the blue is less used in commercial PPG applications.

The term Bluetooth refers herein to a wireless communication technology that enables the exchange of data between electronic devices over short distances using radio waves. It was developed to create a secure, low-power, and efficient method for devices to connect without requiring physical cables.

The term external device to a first device refers herein to a second device in a form of a piece of hardware that connects to or communicates with the first device but operates outside thereto.

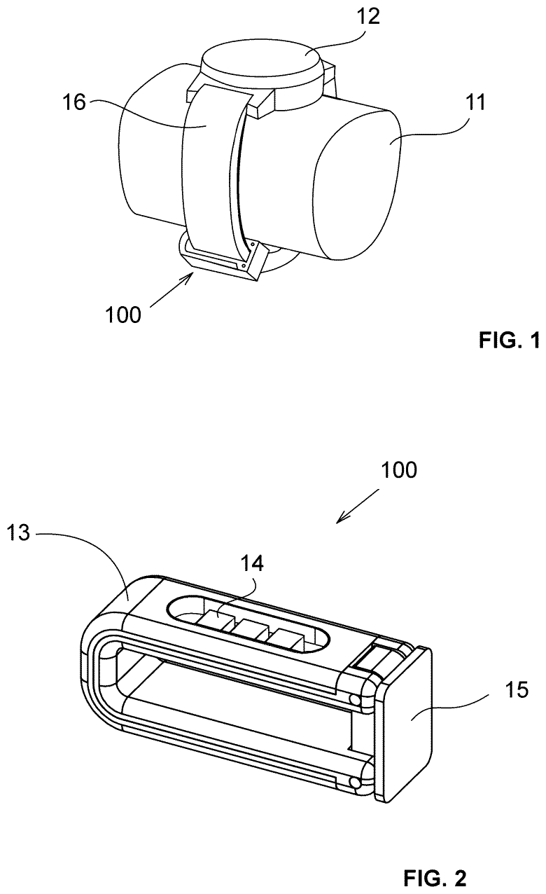

FIG. 1 pictorially illustrates a wrist of a user wearing a wristwatch to which a PPG device is attached, according to an embodiment of the invention.

The PPG device, which is marked herein by reference numeral 100, has an annular shape that allows it to be worn on a strap 16 of wristwatch 12 on a user 11 wrist. Due to the fact that the PPG device 100 surrounds the strap 16 across its width, since the strap 16 comes into contact with the user wrist 11, it also comes into contact with the user's wrist 11, which allows its PPG sensors to sample the user's body. Contact with the user's body is not only essential, but also improves the accuracy of the sampling. The physical size of the PPG device 100 is similar to the physical size of a smart ring, and therefore the PPG device can be given the features of a smart ring.

FIG. 2 schematically illustrates a PPG device, according to an embodiment of the invention.

The PPG device 100 has an annular body 13, in order to allow attaching it to a strap of a wristwatch.

The PPG device may comprise a closure 15; however, the closure is optional.

In the body 13 of the PPG device 100 are embedded one or more PPG sensors 14.

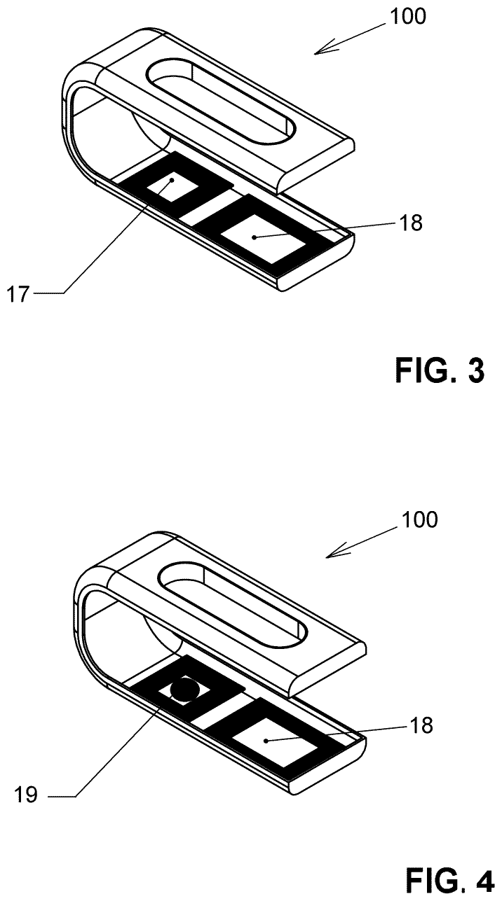

FIG. 3 pictorially illustrates a PPG device, according an embodiment of the invention.

For the sake of clarity, the closure is not shown.

This drawing is a perspective view of a PPG device 100. The drawing shows the inside of the PPG device. As can be seen in the drawing, the inner side contains an NFC antenna 17 for short-range communication, as well as a charging antenna 18 for charging the PPG device.

The PPH device can be designed to contain NFC and/or MCU (i.e., microcontroller) for communication with other devices that can analyze the data the PPG device samples from a user's body.

The choice between NFC and Bluetooth for a PPG-enabled device depends on several factors, including communication range, power requirements, and data transfer rate. The following is a brief comparison of the options:
1. NFC (Near Field Communication):
   Range: Very short range (up to 10 cm);
   Energy requirements: Very low;
   Data transfer rate: Relatively low;
   Uses: Suitable for short-range contact communication, such as mobile payments or touch ID.
2. Bluetooth:
Range: Medium (up to 10-100 meters, depending on the version);
Energy requirements: Low to medium (Bluetooth Low Energy-BLE can be very energy efficient);
Data transfer rate: Medium;
Uses: Suitable for portable applications such as smart watches, fitness bands and other devices that require easy and convenient wireless connectivity.

Of course, the PPG device can be powered also by a battery.

As mentioned, the use of NFC communication allows to carry out contactless payments (such as Apple Pay and Google Wallet), public transportation (smart travel cards), and data sharing between devices (such as photos and information between smartphones). The PPG device can send low-frequency radio signals to transfer information securely and simply, so it is quick and convenient to use.

In the same way, an IMU unit can be added to the PPG device, and so on. IMU stands for Inertial Measurement Unit. It is a device that combines three main sensors: a gyroscope, an accelerometer, and a magnetic field meter. The main use of an IMU is to measure motion and direction.

A gyroscope measures angular velocity, an accelerometer measures linear acceleration, and a magnetometer measures the magnetic field. Combining the data from these sensors allows the position, velocity, and direction of a moving object to be determined.

Combining an IMU (Inertial Measurement Unit) with a PPG (Photoplethysmography) unit can be useful in a variety of medical and sports applications. Here are some examples:
1. Exercise tracking: IMU can measure movement and direction, while PPG can measure heart rate and blood flow. The combination of the two sensors can provide comprehensive information about exercise, including heart rate, movement, speed, and direction;
2. Heart health tracking: IMU can track body movements, while PPG can measure heart rate and blood oxygen level. The combination of the two sensors can provide a more complete picture of the state of the heart and blood vessels;
3. Sleep tracking: IMU can measure movements during sleep, while PPG can measure heart rate and blood oxygen level. The combination of the two sensors can provide information about sleep quality and sleep patterns;
4. Sports tracking: IMU can measure sports movements, while PPG can measure heart rate and blood oxygen level. The combination of the two sensors can provide information about sports performance and recovery after exercise.

In summary, combining IMU and PPG can provide rich and comprehensive information about body movements and health status, and tracking physical activity and heart health of a user.

FIG. 4 pictorially illustrates a PPG device, according an embodiment of the invention.

For the sake of clarity, the closure is not shown.

This drawing is a perspective view of a PPG device 100. The drawing shows the inside of the PPG device. As can be seen in the drawing, the inner side contains a battery 19.

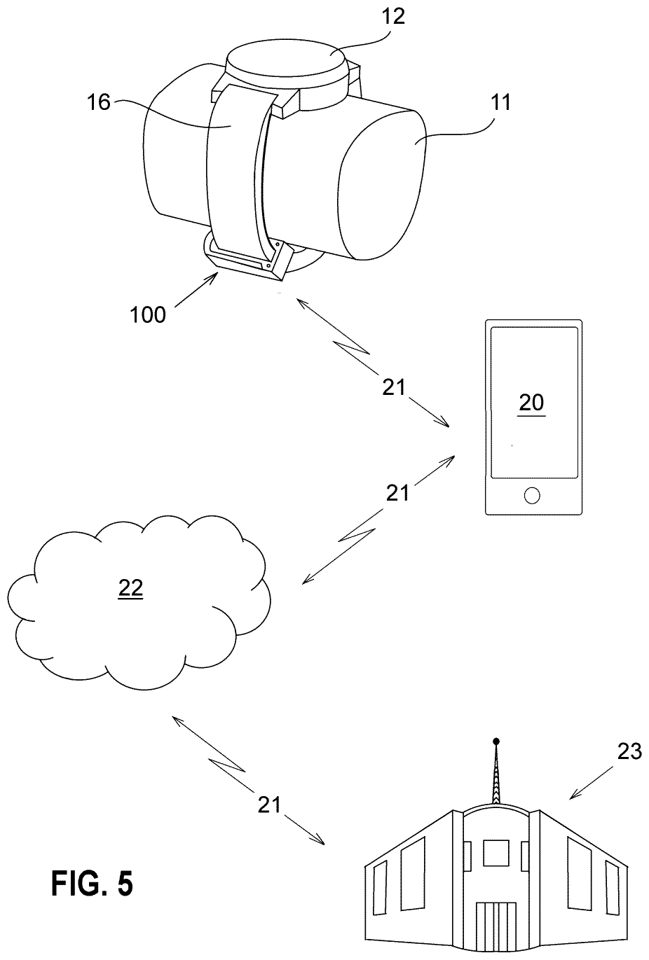

FIG. 5 schematically illustrates a PPG device 100 communication with a smartphone 20, according an embodiment of the invention.

The communication may be of NFC communication, Bluetooth communication, or any wired or wireless communication.

According to a further embodiment of the invention, if the measurements of the PPG sensors indicate a heart attack or another health emergency situation, it can report via the internet 22 to a healthcare institute 23, that can immediately send an ambulance to the patient, or treat the situation in another manner.

In the figures and/or description herein, the following reference numerals (Reference Signs List) have been mentioned:
numeral 11 denotes a user wrist;
numeral 12 denotes a wristwatch;
numeral 13 denotes a body of a PPG device;
numeral 14 denotes a PPG sensor;
numeral 15 denotes a closure of a PPG device, for closing the PPG device around a strap of a wristwatch;
numeral 16 denotes a strap of a wristwatch;
numeral 17 denotes an NFC antenna;
numeral 18 denotes a charging antenna of a PPG device; numeral 19 denotes a battery;
numeral 20 denotes a smartphone; and
numeral 21 denotes wireless communication.

The foregoing description and illustrations of the embodiments of the invention has been presented for the purposes of illustration. It is not intended to be exhaustive or to limit the invention to the above description in any form.

Any term that has been defined above and used in the claims, should to be interpreted according to this definition.

The reference numbers in the claims are not a part of the claims, but rather used for facilitating the reading thereof. These reference numbers should not be interpreted as limiting the claims in any form.

1. A PPG device, comprising:
at least one photoplethysmography (PPG) sensor (14), for measuring health related characteristics of a user thereof;
a charging device, for providing power to said PPG device; and
an output interface, for exporting a reading of said at least one PPG sensor to an external device thereto,
wherein said PPG device is a closed geometric structure having an openable closure (15) and configured to surround a strap (16) of a wristwatch (12), and contain therein said at least one PPG sensor (14),
wherein a size of the hole of said closed geometric structure is suitable to surround said strap (16) of said wristwatch (12).
thereby enabling contact with the user's body for allowing measurement of health related data thereof.

2. (canceled)

3. The PPG device according to claim 1, wherein said PPG sensor tracks heart pulses.

4. The PPG device according to claim 1, wherein said PPG sensor measures stress levels of a user.

5. The PPG device according to claim 1, wherein said PPG sensor measures blood oxygen levels.

6. The PPG device according to claim 1, wherein said output interface employs NFC communication, being a near field communication ability.

7. The PPG device according to claim 6, wherein said NFC communication is used for transferring PPG data out of said PPG device to an external device thereto.

8. The PPG device according to claim 6, wherein said NFC communication is used for carrying out contactless payments to an external device thereto.

9. The PPG device according to claim 1, wherein said output interface employs Bluetooth communication ability.

10. The PPG device according to claim 1, further comprising an IMU sensor, for providing ability to track physical activity and heart health parameters of said user.

11. The PPG device according to claim 1, wherein said external device is a smartphone of said user.

12. The PPG device according to claim 1, wherein a reading of said PPG device is transmitted to a smartphone of said user, and therefrom to a communication device of a medical institute.

13. The PPG device according to claim 1, wherein said charging device is a wireless charging device.

14. The PPG device according to claim 1, wherein said charging device is a wireless charging battery.

15. (canceled)
